SEQUENCE LISTING (2 sequences)

SEQ ID NO: 1 - DNA, 1574 bp, Homo sapiens
gagtagagccgatctcccgcgccccgaggttgctcctctccgaggtctcccgcggcccaagttctccgcgccccgaggtctccgcgccccgaggtctccgcggcccgaggtctccgcccgcaccatgcggctgggcagtcctggactgctcttcctgctcttcagcagccttcgagctgatactcaggagaaggaagtcagagcgatggtaggcagcgacgtggagctcagctgcgcttgccctgaaggaagccgttttgatttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccaggacgagcagaagtttcactgcctggtgttgagccaatccctgggattccaggaggttttgagcgttgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactggccacgtttgaccggagctcaccgcccagagcgtggacagggcttccgtgagacgccaccgtgagaggccaggtggcagcttgagcatggactcccagactgcaggggagcacttggggcagcccccagaaggaccactgctggatcccagggagaacctgctggcgttggctgtgatcctggaatgaggccctttcaaaagcgtcatccacaccaaaggcaaatgtccccaagtgagtgggctccccgctgtcactgccagtcacccacaggaagggactggtgatgggctgtctctacccggagcgtgcgggattcagcaccaggctcttcccagtaccccagacccactgtgggtcttcccgtgggatgcgggatcctgagaccgaagggtgtttggtttaaaaagaagactgggcgtccgctcttccaggacggcctctgtgctgctggggtcacgcgaggctgtttgcaggggacacggtcacaggagctcttctgccctgaacgctcccaacctgcctcccgcccggaagccacaggacccactca

SEQ ID NO: 2 - protein, 302 aa, Homo Sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

(19) United States
(12) Patent Application Publication
(10) Pub. No.: US 2007/0110710 A1
(43) Pub. Date: May 17, 2007

(54) Treatment with immunoregulatory t cells

(75) Inventors: Francine M. Foss, Woodbridge, CT (US); Yong Xie, Belmont, MA (US)

Correspondence Address: FISH & RICHARDSON PC, P.O. BOX 1022, MINNEAPOLIS, MN, 55440-1022, US

(73) Assignee: NEW ENGLAND MEDICAL CENTER HOSPITALS, INC., Boston, MA (US)

(21) Appl. No.: 10/578,797

(22) PCT Filed: Nov. 5, 2004
(86) PCT No.: PCT/US04/37201

§ 371 Date: Oct. 3, 2006

Related U.S. Application Data
(60) Provisional application No. 60/517,624, filed on Nov. 5, 2003.

(51) Int. Cl. A61K 38/19; A61K 35/14

(52) U.S. Cl. 424/85.1; 424/93.7; 607/86

(57) ABSTRACT

Methods and compositions are provided for the treatment of an autoimmune disease. The methods include administering immunoregulatory T cells (T_{regs}) to patients. The therapeutic T_{regs} can express the CD25, CD4, and/or ICOS antigens. The therapeutic T cells can be CD25⁺ ICOS⁺, CD4⁺ICOS⁺, and/or CD25⁺ CD4⁺ ICOS⁺, and they can be provided by a donor.

### TECHNICAL FIELD

This invention relates to compositions and methods for administering certain cells to patients, and more particularly to administration of immunoregulatory T cells (T_{regs}) expressing inducible costimulatory molecule (ICOS).

### BACKGROUND

Immunotherapy is based on the idea that the body's own natural defenses can be used to fight disease; many immunotherapies stimulate the immune system either locally or systemically. Such therapies have been proposed for the treatment of autoimmune diseases.

Graft Versus Host Disease (GVHD) is an autoimmune disease that occurs when immunologically competent cells are introduced into an immunoincompetent host, and it occurs frequently in recipients of solid organ transplants. A variety of treatments are available for GVHD, which are effective to varying degrees. The most common treatment involves administration of corticosteroids. However, some patients experience a steroid-refractory GVHD and show little or no improvement in response to this type of treatment. Extracorporeal photophoresis (ECP) is a therapeutic intervention that has demonstrated efficacy in patients with steroid-refractory acute and chronic GVHD.

Cutaneous T_cell lymphoma is a cancer of helper T-cells. Extracorporeal photophoresis is a treatment that has been shown to be effective for the treatment of this disease.

### SUMMARY

In accordance with our interest in providing more effective treatments for individuals suffering from autoimmune disorders, we set out to understand the effects of certain known therapies on the immune system. We discovered that certain populations of T_{regs} shift upon treatment with ECP. We also discovered that the ICOS protein is a marker for cells that are T_{reg}. For example, we discovered that the number of ICOS⁺ CD4⁺ T cells that also express CD25 antigen was increased in some GVHD patients, and patients who exhibited this increase were more likely to benefit from continued ECP treatment than patients whose ICOS⁺ CD4⁺ T cells did not exhibit an increase in CD25 presentation. Accordingly, the present invention features compositions and methods for treating patients who have, or who are at risk for developing, autoimmune diseases such as GVHD and others described below, and certain cancers which have shown benefit from immunomodulatory treatments, such as cutaneous T-cell lymphoma.

ECP is an immunomodulatory technique based on pheresis of light-sensitive cells. Typically, an ECP therapy includes leukapheresis to isolate leukocytes from a patient. The leukocytes are exposed to a photosensitizing agent, such as psoralen (e.g., 8-methoxypsoralen (8-MOP)), and then the leukocytes are exposed to ultraviolet-A (UVA) light. The irradiated leukocytes are then returned to the patient. ECP therapy has been found to be an effective treatment for many autoimmune diseases, including GVHD. ECP has also been shown to be an effective treatment for the lymphocytic cancer, cutaneous T-cell lymphoma.

While we describe the compositions and methods further below, we note here that the compositions include physiologically acceptable (e.g., non-toxic or substantially non-toxic) compositions that contain ICOS⁺ CD4⁺ T cells that also express CD25 antigen, and the methods include methods of administering those compositions to a patient in need thereof (e.g., a human patient who has received or who is scheduled to receive a transplant or a patient who has, or who is at risk for developing, an autoimmune disease (such patients include patients who are suffering from GVHD that has proven refractory to another treatment)). The methods can be carried out, for example, by identifying an individual (e.g., a human patient) who has been diagnosed as having an autoimmune disease and administering to that individual a purified population of ICOS⁺ CD25⁺ (and/or ICOS⁺ CD4⁺ and/or ICOS⁺ CD25⁺ CD4⁺) cells. A purified population of cells includes less than about 10% (e.g., less than about 8%, 6%, 5% or 3%) of cells that are not of the specified type. A purified population of cells typically includes about 10¹⁰-10¹¹ cells for administration to the human. The cells can be maintained in or obtained from cell culture or they can be harvested from a variety of sources (including the patient, a relative of the patient, or an unrelated donor). The cells can be maintained in a purified state.

The cells can be administered in the same manner that any cells used in cell-based therapies are presently administered to a patient. Cell-based therapies are described, for example in U.S. Patent Publications 2003/0223968 and 2004/0215334. Generally, the number of cells and the frequency with which they are administered (whether once or on multiple occasions) will be sufficient to improve the patient's prognosis. Moreover, based on that prognosis, a patient and his or her physician can better determine whether a course of therapy (e.g., ECP) should be continued or replaced by, or supplemented by, another therapy. The aim of the cellular administration is to enhance the patient's immune response and enable the patient to fight the autoimmune disease or cancer more effectively. Evidence of improvement may come in the form of improvement of an objective sign of the disease or the patient's subjective report of an improvement (e.g., alleviated symptoms).

Alternatively, or in addition, a patient diagnosed as having, or considered to be at risk for developing, an autoimmune disease can be treated with a ligand of ICOS. These ligands are known in the art and include B7H/B7RP1 and a B7H2 polypeptide (see FIG. 1). Fragments of the ligand that retain the ability of the ligand to bind ICOS can also be used. Binding assays can include ELISA assays, Far Western assays, bead based assays, and immunocytochemistry techniques. These ligands can be administered to a patient directly (e.g., formulated in an injectable, physiologically acceptable carrier) or indirectly (e.g., one can administer a nucleic acid sequence that encodes the ligand; for example, a sequence contained within an expression vector). Alternatively, or in addition, the patient can be treated with a cell that expresses a ligand of ICOS. While the methods of the invention may or may not be carried out on a cellular level in the way we suspect, our current theory is that ICOS ligands stimulate production of ICOS-expressing T cells. This would skew the population of ICOS expressing cells to populations of ICOS⁺ CD25⁺ cells or ICOS⁺ CD25⁺ CD4⁺ cells and increase the likelihood that the patient will respond positively (e.g., respond more positively to ECP therapy). The aim is to improve the disease, such as by using the cells alone, or to augment the effects of other treatments, such as ECP, antibody therapy, cytokine therapy, or drug therapy.

The methods described herein can be suitably carried out in patients who are diagnosed as having an autoimmune disease, such as GVHD (e.g., acute or chronic GVHD (aGVHD or cGVHD, respectively), or a steroid-refractory GVHD). Patients with other autoimmune diseases can be treated as well. For example, a patient can be diagnosed as having, or of being at risk for developing: (1) a rheumatic disease such as rheumatoid arthritis, systemic lupus erythematosus, Sjögren's syndrome, scleroderma, mixed connective tissue disease, dermatomyositis, polymyositis, Reiter's syndrome or Behcet's disease; (2) type I (insulin dependent) or type II diabetes mellitus; (3) an autoimmune disease of the thyroid, such as Hashimoto's thyroiditis or Graves' Disease; (4) an autoimmune disease of the central nervous system, such as multiple sclerosis, myasthenia gravis, or encephalomyelitis; (5) a variety of phemphigus, such as phemphigus vulgaris, phemphigus vegetans, phemphigus foliaceus, Senead-Usher syndrome, or Brazilian phemphigus; (6) psoriasis (e.g., psoriasis vulgaris) or atopic dermatitis; (7) inflammatory bowel disease (e.g., ulcerative colitis or Crohn's Disease); or (8) a disorder resulting from an organ, tissue, or cell transplant (e.g., a bone marrow transplant), such as acute or chronic GVHD (as stated above), or Aplastic Anaemia. The T_{regs} described herein can be used to treat other autoimmune disorders including, but not limited to, endogenous uveitis, nephrotic syndrome, primary biliary cirrhosis, lichen planus, pyoderma gangrenosum, alopecia areata, a Bullous disorder, chronic viral active hepatitis, autoimmune chronic active hepatitis, and acquired immune deficiency syndrome (AIDS). In addition, patients who have received a vascular injury would benefit from the methods described herein. We have noted that individuals who are at risk of developing an autoimmune disease are also candidates; these individuals include transplant recipients (i.e., any patient who is scheduled to receive a whole organ, a tissue or a population of cells (e.g., stem cells)).

The methods featured in the invention can also be carried out in patients who are diagnosed as having a cancer, such as a lymphocytic cancer, such as cutaneous T cell lymphoma, a disease in which ECP is known to induce an increase in populations of T_{regs}. Lymphocytic cancer results when T cells of the lymphatic system (also called T-lymphocytes) become malignant and affect the skin. The use of cell based therapies that include administration of T_{regs} such as those described herein are included. The methods can also be used to maintain a response or to prevent relapse of the disease.

Any of the methods described above (or herein) can include an additional step in which the patient is monitored in order to determine whether the treatment has had an effect (whether desirable or undesirable) on their condition or whether the symptoms of their disorder have improved.

Any of the methods described above (or herein) can be carried out in conjunction with another therapy, such as an ECP therapy or one involving administration of an immunosuppressive agent (e.g., a drug such as cyclosporin) or an antibody therapy, such as etanercept or infliximab or rituximab, or a cytotoxic chemotherapy agent or regimen, or a biomodulatory therapy, such as interferon alfa. In addition, the patient can receive one or more agents to combat related or secondary infections (e.g., an antibiotic antifungal, or antiviral agent) or to relieve pain or inflammation (e.g., aspirin or a non-aspirin pain reliever).

As noted above, the methods featured in the invention have prognostic as well as therapeutic value. For example, one can determine whether a patient is likely to benefit from ECP therapy or from a therapy for cancer by determining whether the treatment is affecting the patient's T cell population. For example, one can determine whether there are any changes in the levels of T cell subpopulations, including the subpopulations of CD25⁺ ICOS⁺, CD4⁺ ICOS⁺, and/or CD25⁺ CD4⁺ ICOS⁺ cells. If the patient experiences an increase in any of these cell populations following the initial ECP therapy, then the patient is likely to have a positive response to further ECP therapy. If the patient does not demonstrate an increase in any one of the CD25⁺ ICOS⁺, CD4⁺ ICOS⁺, or CD25⁺ CD4⁺ ICOS⁺ cell populations following the initial ECP therapy, or if the patient demonstrates a decrease in T_{regs}, then it is not likely that the patient will benefit from additional ECP treatments. At that point, alternative treatments may be pursued. An initial ECP therapy can be, for example, 1, 2, or 3 ECP treatments, or the minimal number of ECP treatments typically required to elicit a T cell response.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, useful methods and materials are described below. The materials, methods, and example are illustrative only and are not intended to be limiting. Other features and advantages of the invention will be apparent from the description and the claims.

### BRIEF DESCRIPTION OF THE DRAWING

FIG. 1 is a description of the human B7-like protein B7-H2, which can be used as an ICOS ligand; the nucleotide and amino acid sequences are shown.

### DETAILED DESCRIPTION

The present invention provides methods and compositions for treating a human diagnosed as having, or at risk for developing, an autoimmune disease. One exemplary method includes treating a human, such as a human patient, with a therapeutic composition that includes T_{regs}, and particularly T_{regs} that express the CD25, CD4, and/or ICOS antigens. The therapeutic T cells can be CD25⁺ ICOS⁺, CD4⁺ ICOS⁺, and/or CD25⁺ CD4⁺ ICOS⁺, and they can be provided by a donor (who may be the patient, a genetic relative of the patient, or an unknown individual). A donor of the T_{regs} described herein can be previously known or can have been determined to have a high ratio of CD25⁺ ICOS⁺, CD4⁺ ICOS⁺, and/or CD25⁺ CD4⁺ ICOS⁺ cells, or the donor can be induced to express the cells, such as by administration of one or more ECP treatments.

Indications Exemplary recipients of the therapeutic methods and compositions described herein are subjects who are diagnosed as having an autoimmune disease (specific diseases are listed above) or a cancer, or who are at risk for developing such diseases. For example, a human who has received, or who is scheduled to receive, a tissue graft, organ transplant, blood transfusion, hematopoietic stem cell transplant (HSCT), or the like is at risk for developing an autoimmune disease, such as GVHD (e.g., acute or chronic GVHD, or steroid-refractory GVHD), and is a candidate recipient of the therapeutic methods and compositions described herein.

GVHD occurs when immunologically competent cells are introduced into an immunoincompetent host. GVHD refers to both the immunologic assault and the consequences to the organism. Acute GVHD (aGVHD) occurs within the first 100 days of a transplant and consists of the triad of dermatitis, enteritis, and hepatitis. Chronic GVHD (cGVHD) develops after day 100 and consists of an autoimmune syndrome directed toward multiple organs. Steroid-refractory GVHD refers to GVHD that shows little or no improvement in response to treatment with corticosteroids, the most common treatment for the disease.

Humans diagnosed or at risk for developing other autoimmune disorders can also receive the described therapeutic regimens. For example, subjects diagnosed with or at risk for developing a non-Hodgkin's lymphoma, such as cutaneous T-cell lymphoma (CTCL) (e.g., Sézary syndrome); progressive systemic sclerosis (scleroderma); an autoimmune bullous (blistering) disease, such as pemphigus vulgaris, pemphigus foliaceus, or bullous pemphigoid; systemic lupus erythematosus; multiple sclerosis; psoriatic arthritis or psoriasis vulgaris; rheumatoid arthritis; type I diabetes; atopic dermatitis; juvenile dermatomyositis; or scleromyxedema are candidates for the described therapies.

A human who is diagnosed as having or is at risk for developing a cancer is also a candidate recipient of the therapeutic methods and compositions described herein. The cancer can be any proliferative disorder, and in particular a proliferative disorder that has demonstrated a response to ECP therapy. For example, the human can be diagnosed as having or at risk for developing a lymphocytic cancer such as cutaneous T cell lymphoma. A human suitable for the therapeutic methods featured in the invention may be diagnosed with an acute lymphocytic leukemia, such as acute lymphocytic leukemia (ALL), acute myeloid leukemia (AML), chronic lymphocytic leukemia (CLL), or chronic myelogenous leukemia (CML). Alternatively, the human may be diagnosed as having chronic lymphocytic leukemia or hairy cell leukemia (HCL).

Generation of Immunoregulatory T cells Immunoregulatory T cell production can be stimulated in a human, for example, by ECP therapy. ECP is more typically used as a therapeutic intervention that has demonstrated efficacy in patients with steroid-refractory acute and chronic GVHD. Clinical response in patients with extensive, refractory cGVHD has been associated with normalization of skewed CD4/CD8 ratios and a shift in dendritic cell populations, favoring a DC2/Th2 cytokine profile.

I. Without being bound by theory, administration of an ECP therapy to a human, either a human having an autoimmune disease, such as GVHD, or a healthy human (a human who does not suffer from an autoimmune disease) can increase the levels (and ratios) of CD4⁺ T cells. Further, administration of ECP can increase the amount of ICOS⁺ CD4⁺ cells and/or ICOS⁺ CD4⁺ CD25⁺ cells, and can increase the ratio of ICOS⁺ CD25⁺/ICOS⁻ CD25⁺ cells in a human. The increase in the levels of these T cell populations can decrease the symptoms caused by an autoimmune disease. Thus administration of ICOS⁺ CD25⁺, ICOS⁺ CD4⁺, and/or ICOS⁺ CD25⁺ CD4⁺ to a human suffering from an autoimmune disease or at risk for developing an autoimmune disease can be an effective therapy against the disease.

The ICOS⁺ CD25⁺, ICOS⁺ CD4⁺, and ICOS⁺CD25⁺ CD4⁺ cells can be isolated from a donor person determined to have high levels of these cell types. The levels of these cell types can be determined by a variety of immunocytochemistry methods known in the art, including but not limited to the ELISA and EliSpot assays (Czerzinsky et al., J. Immunol. Meth. 65:109, 1983). Expression of these cell types in a donor subject can be induced by treatment with ECP. For example, a donor person can be any human known or unknown to the intended recipient. The donor can be a relative of the intended recipient, or the donor can be the recipient himself (or herself). For example, ICOS⁺CD25⁺, ICOS⁺ CD4⁺, and ICOS⁺ CD25⁺ CD4⁺ cells can be isolated from an intended recipient and stored for later use. For example, the cells can be harvested from the recipient before he/she receives a tissue graft or organ transplant.

Alternatively or in addition to harvesting T_{regs} from a donor, the immunotherapeutic cells can be expanded, such as in culture, before their administration to a patient, or tissue or organ. To expand the number of cells, the culture medium can contain necessary nutrients and components known in the art to be necessary for division of cells expressing ICOS, CD25, and CD4. It is not necessary that the medium contain components that selectively encourage the division of any of the described preferred cell types (e.g., ICOS⁺ CD25⁺, ICOS⁺ CD4⁺, or ICOS⁺ CD25⁺ CD4⁺ cells). The medium can include, for example, dendritic cells (DC) (with or without antigen), cytokines (e.g., IL-2), or an ICOS ligand, such as B7H2. The cells can be monitored for expression of ICOS, and the CD4 and CD25 antigens, and the desired cell types can be harvested by methods known in the art.

The cells can be isolated from a donor for immediate use for treatment purposes, or the cells can be stored for later use. The storage methods and practices appropriate for maintaining cell viability and/or biological activity are known in the art. As used herein, biological activity refers to the in vivo activities of immune cells or physiological responses that result upon in vivo administration of a cell, composition or other mixture. Biological activity therefore encompasses therapeutic effects and pharmaceutical activities of such cells, compositions and mixtures.

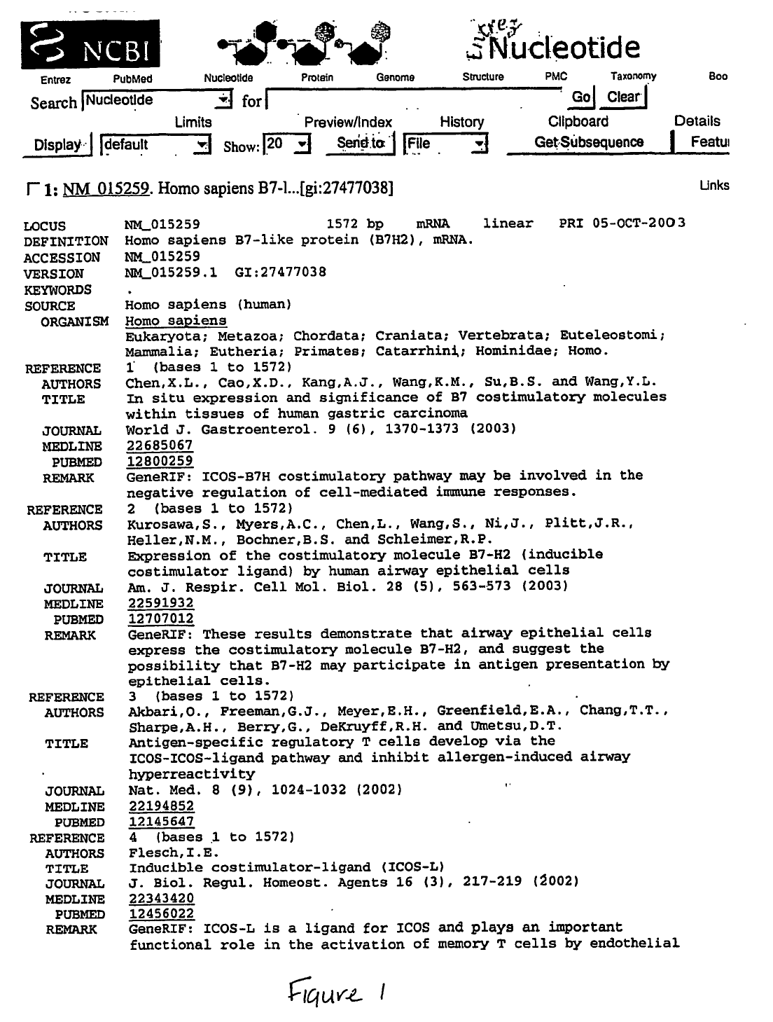

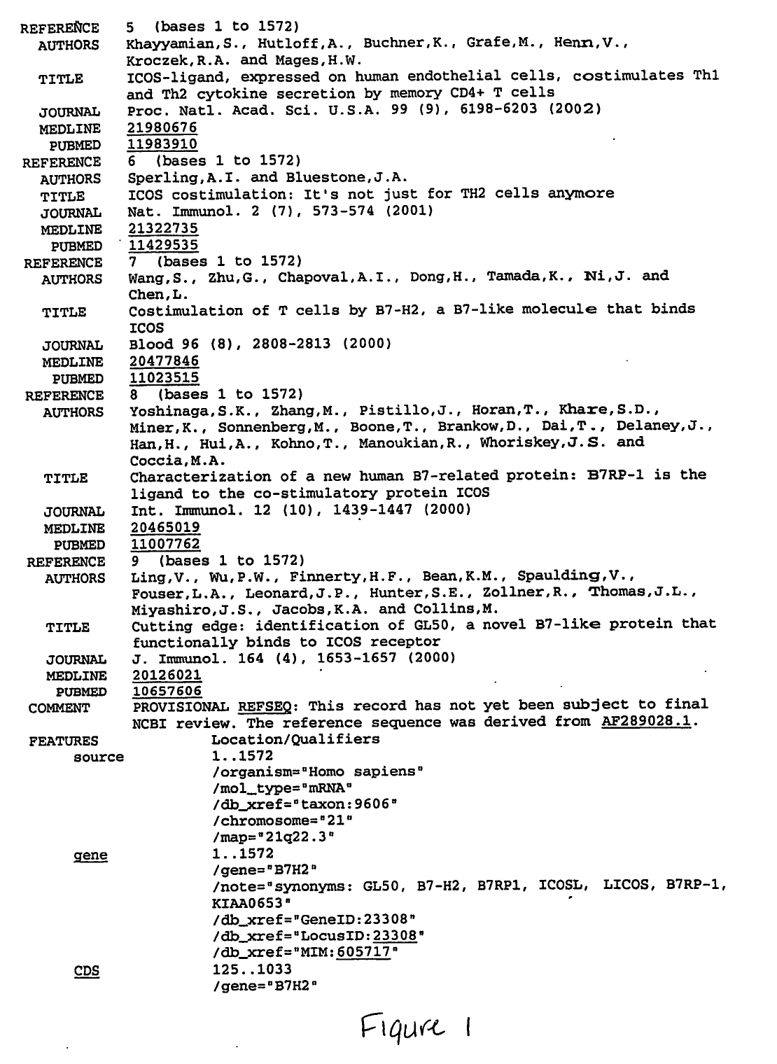

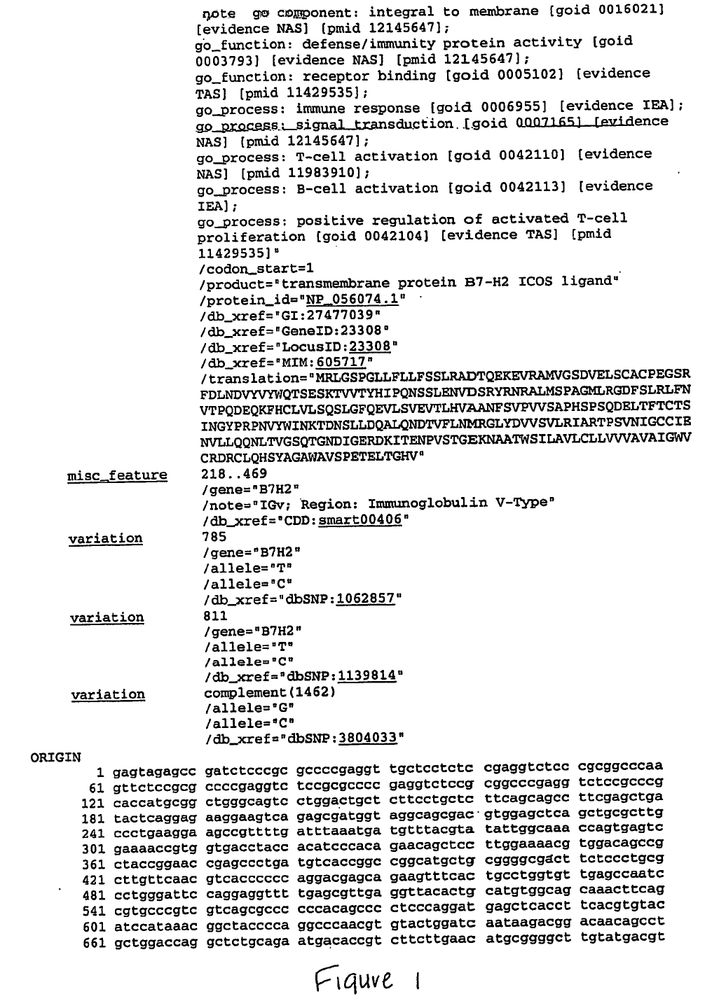

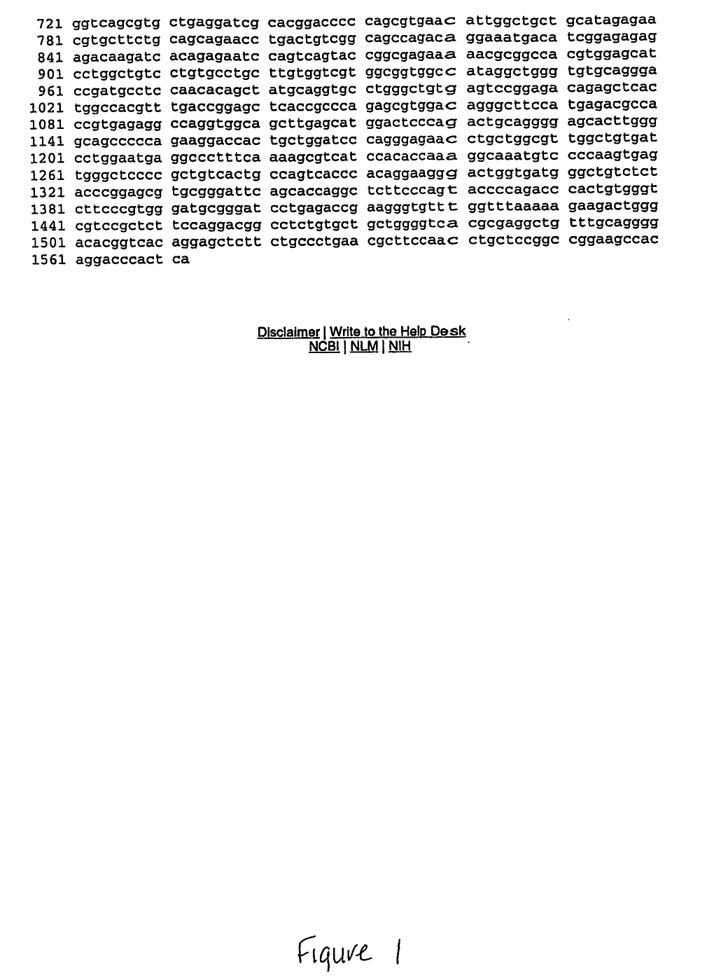

An ICOS Ligand to Treat an Autoimmune Disease The invention disclosed herein also includes a method of treating a human diagnosed as having or at risk for developing an autoimmune disease with a ligand of ICOS, such as a B7H/B7RP1 or B7H2 polypeptide or polypeptide fragment (FIG. 1). The protein can be administered directly, or by means of a nucleic acid vector, such as by gene therapy. A nucleic acid vector, for example, can encode and express the ICOS ligand, and so elicit a therapeutic effect.

While not being bound by theory, administration of an ICOS ligand to a subject can stimulate the production of ICOS⁺ T cells, including the populations described herein (e.g., ICOS⁺ CD25⁺, ICOS⁺ CD4⁺, and ICOS⁺ CD25⁺ CD4⁺ cells), and thereby effectively treating the subject by increasing levels of immunotherapeutic T cells.

Predictive Medicine Also provided are methods of assessing a patient for an appropriate treatment of an autoimmune disease. One such method includes administering an ECP therapy to a patient, such as a GVHD patient, and examining the effects of the treatment on levels of CD25⁺ ICOS⁺, CD4⁺ ICOS⁺, and/or CD25⁺ CD4⁺ ICOS⁺ cells. If the patient demonstrates an increase in any of these cell populations following the initial ECP therapy, then the patient is determined to be likely to have a positive response to further ECP therapy. If the patient does not demonstrate an increase in any one of the CD25⁺ ICOS⁺ or CD4⁺ ICOS⁺ cell populations following the initial BCP therapy, then it is determined that the patient is not likely to benefit from additional ECP treatments, and alternative treatments may be pursued. An initial ECP therapy can be, for example, 1, 2, or 3 ECP treatments, or the minimal number of ECP treatments required to elicit a T cell response in patients who will elicit a response.

These methods can be applied to patients with cancer, such as a lymphocytic cancer, such as cutaneous T cell lymphoma, where the levels of T_{regs} can be measured by CD25⁺ CD4⁺ ICOS⁺ cell populations and correlated with clinical response to therapy. For instance, response to interleukin-2 based cytokine therapy in patients with malignant melanoma is associated with suppression of tolerogenic immunoregulatory T-cells. A decrease in T_{regs}, such as those described herein, may be correlated with clinical response to a given therapy and may be a marker of disease progression.

Formulations and Routes of Administration The therapeutic compositions described herein (e.g., those containing the specified T_{regs} or compositions that induce ICOS expression or that bind ICOS) can be administered in a variety of formulations. For example, the T_{regs} can be administered at various degrees of purity. The ICOS⁺ CD25⁺, ICOS⁺ CD4⁺, and ICOS⁺ CD25⁺ CD4⁺ cells can be administered together as a heterogeneous mix, and other cell types can be present in the mix.

Methods for purification of the immune cells to produce substantially pure populations (e.g., substantially pure populations of each or combinations of ICOS⁺ CD25⁺, ICOS⁺ CD4⁺, ICOS⁺ CD25⁺ CD4⁺ cell populations) are known to those of skill in the art. A substantially pure cell population, may, however, be a mixture of subtypes; purity refers to the activity profile of the population. In such instances, further purification might increase the specific activity of the cell population.

Administration of the therapies described herein can be performed by a variety of methods known in the art. For example, the administration of the T_{regs} can be by the general method of cell therapy, which includes the administration of live cells. The T_{regs} can be administered by any suitable means, including, but not limited to, intravenously, parenterally, or locally. The particular mode selected will depend upon the particular treatment and trafficking of the cells. Typically, about 10¹⁰-10¹¹ cells can be administered in a volume of a 50 ml to 1 liter, 50 ml to 250 ml, 50 ml to 150, and typically 100 ml. The volume will depend upon the targeted disorder and the route of administration. The cells can be administered in a single dose or in several doses over selected time intervals in order to titrate the dose.

A human who is administered therapeutic T_{regs}, can also receive at least a second therapeutic regimen, such as an ECP therapy, antibody or biological therapy, cytokine therapy, cytotoxic chemotherapy, or an immunosuppressive drug. Immunosuppressant drugs include corticosteroids (e.g., glucocorticoids such as methylprednisolone), cyclosporine, FK506, mycophenolate mofetil (MMF), and antithymocyte globulin (ATG). The use of high-dose corticosteroids can increase the risk of opportunistic infections, and thus concomitant prophylactic antibiotic, antiviral, and antifungal therapy can also be administered.

A transplant tissue or organ can be treated with any of the therapeutic compositions described herein before delivery to a recipient. For example, the therapeutic T_{regs} (e.g., the purified ICOS⁺ CD25⁺, ICOS⁺ CD4⁺, or ICOS⁺ CD25⁺ CD4⁺ cells, or a combination of the three cell types) can be administered to the tissue or organ. Administration can be by any suitable method known in the art, such as by bathing the tissue or organ in a solution containing the cells or by injecting the cells into the tissue or organ.

Following administration of a therapeutic composition described herein, the patient can be monitored for an improvement in the symptoms or the severity of the autoimmune disorder, or for the development of an autoimmune disorder, such as in the time period following an organ transplant.

The invention is further illustrated by the following example, which should not be construed as further limiting. The contents of all references, pending patent applications and published patents, cited throughout this application are hereby expressly incorporated by reference. In case of conflict, the present specification, including definitions, will control.

### II. EXAMPLE

Clinical response to ECP in patients with extensive, refractory GVHD was associated with normalization of skewed CD4/CD8 ratios and a shift in dendritic cell (DC) populations, favoring a DC2/T helper 2 (Th2) cytokine profile. ICOS is a member of the B7-CD28 superfamily, is expressed by activated T-lymphocytes, and is involved in T cell activation, IL-10 production and Th1/Th2 differentiation. In murine models, anti-ICOS antibodies have been shown to have opposing effects, attenuating the manifestations of chronic Graft versus Host Disease (cGVHD) and exacerbating the symptoms of acute GVHD (aGVHD).

ECP Therapy Increased ICOS⁺ CD4⁺ CD25⁺ T cell Populations and Correlated with Effective Treatment of GVHD with ECP Twelve patients undergoing ECP treatment for cGVHD were examined for ICOS expression on various T cell populations, including CD4, CD8, CD25, and CD69 positive populations. All patients received ECP therapy for two consecutive days every other week or weekly. Peripheral blood mononuclear cells were examined at baseline and after 2-3 months of ECP therapy for expression of ICOS on CD4⁺ and CD8⁺ T-cells. At least a 2-fold increase in the number of ICOS⁺ CD4⁺ T-cells that co-expressed CD25 was observed in seven patients, and these seven patients each also had a positive response to ECP. Generally, more ECP treatments generated more CD25⁺ ICOS⁺ cells and clinical response was associated with an increase in the number of CD4⁺ ICOS⁺ cells and an increase in the ratio of CD25⁺ ICOS⁺ cells to CD25⁺ ICOS⁻ cells. Of four patients in whom the ratio of CD25⁺ ICOS⁺ cells to CD25⁺ ICOS⁻ cells decreased, or was unchanged, none responded to ECP.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating a patient diagnosed as having, or at risk for developing, an autoimmune or proliferative disease, the method comprising:
(i) providing a purified population of CD25⁺ ICOS⁺ cells from a donor, and
(ii) administering the cells to the patient in an amount and for a time sufficient to improve a sign or symptom of the autoimmune disease in the patient.

2. The method of claim 1, wherein some or all of the CD25⁺ ICOS⁺cells are also CD4⁺.

3. The method of claim 1, wherein the patient is diagnosed as having, or is at risk for developing, graft versus host disease (GVHD).

4. The method of claim 3, wherein the GVHD is acute GVHD.

5. The method of claim 3, wherein the GVHD is chronic GVHD.

6. The method of claim 3, wherein the GVHD is a steroid-refractory GVHD.

7. The method of claim 1, wherein the patient is diagnosed as having, or is at risk for developing, a cancer.

8. The method of claim 7, wherein the cancer is a lymphocytic cancer.

9. The method of claim 8, wherein the lymphocytic cancer is cutaneous T cell lymphoma.

10. The method of claim 1, wherein the patient receives an ECP therapy prior to step (i).

11. The method of claim 1, wherein the patient has received, or is scheduled to receive, a transplant.

12. The method of claim 1, wherein the patient is further administered a second therapeutic regimen.

13. The method of claim 12, wherein the second therapeutic regimen comprises an ECP therapy, an antibody, a cytokine therapy, or a chemotherapy.

14. The method of claim 12, wherein the second therapeutic regimen comprises an immunosuppressive drug.

15. The method of claim 1, further comprising monitoring the patient for an indication that a sign or symptom associated with the autoimmune disorder has been alleviated.

16. A composition comprising a purified population CD25⁺ ICOS⁺ cells provided from a donor, wherein the donor received an ECP therapy prior to providing the CD25⁺ ICOS⁺ cells.

17. A method of treating a patient diagnosed as having, or at risk for developing, an autoimmune disorder, the method comprising administering to the patient a population of cells expressing an ICOS ligand to the human.

18. The method of claim 17, wherein the ICOS ligand is a B7H or B7H2 polypeptide.

19. A method of increasing ICOS⁺ T cell levels in a mammal, the method comprising administering an ICOS ligand to the mammal.

20. The method of claim 19, wherein the ICOS ligand is a B7H or B7H2 polypeptide.

21. The method of claim 19, wherein the mammal is a human.

22. A method of treating a patient diagnosed as having, or at risk for developing, an autoimmune disorder, the method comprising:
(i) providing CD4⁺ ICOS⁺ cells purified from a donor, and
(ii) administering the isolated cells to the patient.

23. A method of assessing a patient for an appropriate treatment of an autoimmune disorder, the method comprising:
(i) administering an ECP therapy to the patient;
(ii) determining that (A) if the patient demonstrates an increase in ICOS⁺ CD25⁺ cells following the ECP therapy, then the patient is likely to have a positive response to further ECP therapy; and (B) if the patient does not demonstrate an increase in ICOS⁺ CD25⁺ cells following the ECP therapy, then further ECP treatments are not likely to reduce the symptoms of the autoimmune disorder, thereby assessing the patient for an appropriate therapy.

24. A pharmaceutically acceptable or physiologically compatible composition comprising purified ICOS⁺ CD25⁺ T cells and/or ICOS⁺ CD4⁺ and/or ICOS⁺ CD25⁺ CD4⁺ T cells and a pharmaceutically acceptable carrier.
